(19) United States
(12) Patent Application Publication
(10) Pub. No.: US 2005/0171190 A1
(43) Pub. Date: Aug. 4, 2005

(54) Substituted 5-chroman-5-YL-ethylamine compounds and their use for the treatment of glaucoma

(75) Inventors: Jesse A. May, Fort Worth, TX (US); Anura P. Dantanarayana, MwKandy (LK)

Correspondence Address: KILYK & BOWERSOX, P.L.L.C., 53 A EAST LEE STREET, WARRENTON, VA, 20186, US

(73) Assignee: Alcon, Inc.

(21) Appl. No.: 11/063,286

(22) Filed: Feb. 23, 2005

Related U.S. Application Data
(63) Continuation of application No. PCT/US03/26886, filed on Aug. 27, 2003.
(60) Provisional application No. 60/407,210, filed on Aug. 30, 2002.

(51) Int. Cl. C07D 49/02; C07D 311/74; A61K 31/343; A61K 31/353; A61K 31/381

(52) U.S. Cl. 514/450; 514/456; 549/405; 514/469; 549/467; 514/445; 549/60

(57) ABSTRACT

Substituted 5-chroman-5-yl-ethylamine compounds are disclosed. Also disclosed are methods for the lowering and controlling of normal or elevated intraocular pressure as well as a method for the treatment of glaucoma using compositions containing one or more of the compounds of the present invention.

This application is a continuation of International Patent Application No. PCT/US2003/26886 filed Aug. 27, 2003, which in turn claims the benefit of U.S. Provisional Patent Application No. 60/407,210 filed Aug. 30, 2002, and is incorporated in its entirety by reference herein.

### BACKGROUND OF THE INVENTION

The present invention relates to substituted 5-chroman-5-yl-ethylamine compounds. These novel compounds are useful for lowering and controlling normal or elevated intraocular pressure (IOP) and for treating glaucoma.

The disease state referred to as glaucoma is characterized by a permanent loss of visual function due to irreversible damage to the optic nerve. The several morphologically or functionally distinct types of glaucoma are typically characterized by elevated IOP, which is considered to be causally related to the pathological course of the disease. Ocular hypertension is a condition wherein intraocular pressure is elevated but no apparent loss of visual function has occurred; such patients are considered to be a high risk for the eventual development of the visual loss associated with glaucoma. If glaucoma or ocular hypertension is detected early and treated promptly with medications that effectively reduce elevated intraocular pressure, loss of visual function or its progressive deterioration can generally be ameliorated. Drug therapies that have proven to be effective for the reduction of intraocular pressure include both agents that decrease aqueous humor production and agents that increase the outflow facility. Such therapies are in general administered by one of two possible routes, topically (direct application to the eye) or orally.

There are some individuals who do not respond well when treated with certain existing glaucoma therapies. There is, therefore, a need for other topical therapeutic agents that control IOP.

Serotonergic 5-HT_{1A} agonists have been reported as being neuroprotective in animal models and many of these agents have been evaluated for the treatment of acute stroke among other indications. This class of compounds has been mentioned for the treatment of glaucoma (lowering and controlling IOP), see e.g., WO 98/18458 and EP 0771563A2. Osborne et al. (1996) teach that 8-hydroxy-2-(di-n-propylamino)tetralin (8-OH-DPAT) (a 5-HT_{1A} agonist) reduces IOP in rabbits. Wang et al. (1997 and 1998) indicate that 5-methylurapidil, an α_{1A} antagonist and 5-HT_{1A} agonist lowers IOP in the monkey, but due to its α_{1A} receptor activity. Also, 5-HT_{1A} antagonists are disclosed as being useful for the treatment of glaucoma (elevated IOP) (e.g., WO 92/0338). Furthermore, WO 97/35579 and U.S. Pat. No. 5,578,612 relate to the use of 5-HT₁ and 5-HT₁₋ₗᵢₖₑ agonists for the treatment of glaucoma (elevated IOP). These anti-migraine compounds are 5-HT_{1B,D,E,F} agonists, e.g., sumatriptan and naratriptan and related compounds.

It has been found that serotonergic compounds which possess agonist activity at 5-HT₂ receptors effectively lower and control normal and elevated IOP and are useful for treating glaucoma, see commonly owned co-pending application, PCT/US99/19888, incorporated in its entirety by reference herein. Compounds that act as agonists at 5-HT₂ receptors are well known and have shown a variety of utilities, primarily for disorders or conditions associated with the central nervous system (CNS). U.S. Pat. No. 5,494,928 relates to certain 2-(indol-1-yl)-ethylamine derivatives that are 5-HT_{2C} agonists for the treatment of obsessive compulsive disorder and other CNS derived personality disorders. U.S. Pat. No. 5,571,833 relates to tryptamine derivatives that are 5-HT₂ agonists for the treatment of portal hypertension and migraine. U.S. Pat. No. 5,874,477 relates to a method for treating malaria using 5-HT_{2A/2C} agonists. U.S. Pat. No. 5,902,815 relates to the use of 5-HT_{2A} agonists to prevent adverse effects of NMDA receptor hypo-function. WO 98/31354 relates to 5-HT_{2B} agonists for the treatment of depression and other CNS conditions. U.S. Pat. No. 6,380,238 and International Patent Applications WO 01/12602 and WO 00/44753 relate to indoline derivatives and U.S. Pat. Nos. 6,433,175 and 6,365,598 relate to certain indole derivatives as 5-HT_{2B} and 5-HT_{2C} receptor agonists for the treatment of a variety of disorders of the central nervous system, but especially for the treatment of obesity. WO 00/35922 relates to certain pyrazino[1,2-a]quinoxaline derivates as 5-HT_{2C} agonists for the treatment of obsessive compulsive disorder, depression, eating disorders, and other disorders involving the CNS. WO 00/77002 and WO 00/77010 relate to certain substituted tetracyclic pyrido[4,3-b]indoles as 5-HT_{2C} agonists with utility for the treatment of central nervous system disorders including obesity, anxiety, depression, sleep disorders, cephalic pain, and social phobias among others. Agonist response at the 5-HT_{2A} receptor is reported to be the primary activity responsible for hallucinogenic activity, with some lesser involvement of the 5-HT_{2C} receptor possible (Fiorella et al. 1995).

5-Hyroxytryptamine (serotonin) does not cross the blood-brain barrier and enter the brain. However, in order to increase brain serotonin levels the administration of 5-hydroxy-tryptophane can be employed. The transport of 5-hydroxy-tryptophane into the brain readily occurs, and once in the brain 5-hydroxy-tryptophane is rapidly decarboxylated to provide serotonin. Since the treatment of glaucoma is preferably with compounds that do not enter the CNS, relatively polar compounds that are 5-HT₂ agonists and have incorporated into their structure a phenolic hydroxyl group that can be considered comparable to that of serotonin, are of particular interest.

2-(6,7-Dimethoxy-2,3-dihydro-benzofuran-4-yl)-ethylamine has been synthesized and shown to have affinity for 5-HT2 receptors (Monte et al. 1997).

The preparation of 2-(7-bromo-5-methoxy-2,3-dihydro-benzofuran-4-yl)-1-methylethylamine has been reported (Waldman et al. 1996) and this compound has been shown to have a high affinity for the 5-HT_{2A} receptor and to generalize to LSD in drug discrimination studies (Nichols et al. 1991). Similarly, substituted 5-(2-aminopropyl)-benzodifurans and substituted 5-(2-aminopropyl)-benzodipyrans have been shown to have high affinity for 5-HT₂ receptors and to generalize to LSD in drug discrimination studies (Monte et al. 1996; Parker et al. 1998; Whiteside et al. 2002).

Benzofurans, such as 2-benzofuran-4-yl-1-methylethylamine and related compounds, are reported to have agonist activity at the 5-HT_{2C} receptor and thereby be useful for the treatment of a variety of central nervous system disorders, such as seizure and eating disorders among others (WO 00/44737).

Amides of substituted 3-chroman-5-yl-alkylamines and 3-(2,3-dihydro-benzofuran-4-yl)-alkylamines have agonist or antagonist activity at melatonin receptors and thereby are useful in the treatment of disorders regulated by melatonin. These include chronobiological disorders such as seasonal affective disorders and insomnia, or psychiatric disorders such as bipolar disorders and depression (U.S. Pat. No. 5,981,572).

Accordingly, there is a need to provide new compounds which avoid the disadvantages described above and which provide increased chemical stability and a desired length of therapeutic activity, for instance, in decreasing intraocular pressure and treating glaucoma.

### SUMMARY OF THE PRESENT INVENTION

A feature of the present invention is to provide novel compounds which are 5-HT2 agonists.

Another feature of the present invention is to provide compounds which have increased chemical stability and which are useful in lowering and controlling normal or elevated intraocular pressure and/or treating glaucoma.

Another feature of the present invention is to provide compounds which provide a desired level of therapeutic activity in lowering and controlling normal or elevated intraocular pressure and/or treating glaucoma.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

To achieve these and other advantages, and in accordance with the purposes of the present invention, as embodied and broadly described herein, the present invention relates to a compound having the Formula I:

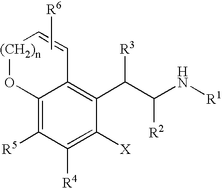

In this formula, R1 is hydrogen or an alkyl group, such as C₁₋₄alkyl;
R² is hydrogen, an alkyl group such as C₁₋₄alkyl, or R¹ and R² can together be (CH₂)₂₋₄ to complete a heterocyclic ring;
R³ is hydrogen, hydroxyl, an alkoxy group such as C₁₋₄alkoxy, or halogen;
R⁴ and R⁵ are independently selected from hydrogen, halogen, nitrile, an alkoxy group such as C₁₋₄alkoxy, an alkylthio such as C₁₋₆alkylthiol, an alkyl group such as C₁₋₄alkyl, a substituted alkyl group such as C₁₋₄alkyl substituted with halogen or C₁₋₆alkoxy; or R⁴ and R⁵ can together be (CH₂)ₘ to complete a cycloalkyl ring, or they can together complete a phenyl or thiophene ring which can be unsubstituted or substituted with halogen, an alkyl group such as C₁₋₄alkyl or an alkoxy group such as C₁₋₄alkoxy;
m=3-4;
n=0-2;
R⁶ is hydrogen, hydroxyl, an alkoxy group such as C₁₋₄alkoxy, a substituted alkoxy such as C₁₋₄alkoxy substituted with hydroxyl, halogen, or NR⁷N⁸, or OC(═O)C₁₋₆alkyl, ═O, NR⁷R⁸, an alkyl group such as C₁₋₄alkyl, a substituted alkyl group such as C₁₋₄alkyl substituted with hydroxyl, halogen, or NR⁷R⁸, however, when n=0, R⁶ cannot be hydrogen;
X is an alkoxy group such as C₁₋₄alkoxy, hydroxyl, or halogen;
R⁷ and R⁸ are independently selected from hydrogen, an alkyl group such as C₁₋₄alkyl, or C(═O)C₁₋₆alkyl; and the dashed bond denotes a single or double bond.

Pharmaceutically acceptable salts and solvates of Formula I are also part of the present invention.

The present invention further relates to pharmaceutical compositions containing at least one compound of Formula I.

The present invention further relates to methods to lower and/or control normal or elevated intraocular pressure by administering an effective amount of a composition containing a compound having Formula I as described above.

The present invention also relates to a method for treating glaucoma which involves administering an effective amount of a composition containing a compound having Formula I as described above.

It is to be understood that both the foregoing general description and the following detailed description are exemplary and explanatory only and are intended to provide a further explanation of the present invention, as claimed.

### DETAILED DESCRIPTION OF THE PRESENT INVENTION

The present invention relates to a variety of compounds which are useful according to the present invention. These compounds are generally represented by the following Formula I.

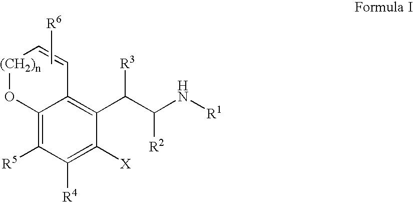

In this formula, R¹ is hydrogen or an alkyl group, such as C₁₋₄alkyl;
R² is hydrogen, an alkyl group such as C₁₋₄alkyl, or R¹ and R² can together be (CH₂)₂₋₄ to complete a heterocyclic ring;
R³ is hydrogen, hydroxyl, an alkoxy group such as C₁₋₄alkoxy, or halogen;
R⁴ and R⁵ are independently selected from hydrogen, halogen, nitrile, an alkoxy group such as C₁₋₄alkoxy, an alkylthio such as C₁₋₆alkylthiol, an alkyl group such as C₁₋₄alkyl, a substituted alkyl group such as C₁₋₄alkyl substituted with halogen or C₁₋₆alkoxy; or R⁴ and R⁵ can together be (CH₂)ₘ to complete a cycloalkyl ring, or they can together complete a phenyl or thiophene ring which can be unsubstituted or substituted with halogen, an alkyl group such as C₁₋₄alkyl or an alkoxy group such as C₁₋₄alkoxy;
m=3-4;
n=0-2;
R⁶ is hydrogen, hydroxyl, an alkoxy group such as C₁₋₄alkoxy, a substituted alkoxy group such as C₁₋₄alkoxy substituted with hydroxyl, halogen, or NR⁷R⁸, OC(═O)C₁₋₆alkyl, ═O, NR⁷R⁸, an alkyl group such as C₁₋₄alkyl, or a substituted alkyl group such as C₁₋₄alkyl substituted with hydroxyl, halogen, or NR⁷R⁸, however, when n=0, R⁶ cannot be hydrogen;
X is an alkoxy group such as C₁₋₄alkoxy, hydroxyl, or halogen;

R⁷ and R⁸ are independently selected from hydrogen, an alkyl group such as C₁₋₄alkyl, or C(═O)C₁₋₆alkyl; and the dashed bond denotes a single or double bond.

Pharmaceutically acceptable salts and solvates of Formula I are also part of the present invention.

Preferred Compounds are:

Wherein R¹ and R³ are hydrogen;
R² is C₁₋₄alkyl;
R⁴ and R⁵ are independently selected from halogen, nitrile, C₁₋₄alkoxy, C₁₋₆alkylthiol, C₁₋₄alkyl, C₁₋₄alkyl substituted with halogen, or R⁴ and R⁵ can together be (CH₂)ₘ to complete a cycloalkyl ring, or they can together complete a phenyl or thiophene ring which can be unsubstituted or substituted with halogen, C₁₋₄alkyl;
m=3-4;
n=1;
R⁶ is hydroxyl, C₁₋₄alkoxy, C₁₋₄alkoxy substituted with hydroxyl, halogen, or NR⁷R⁸, OC(═O)C₁₋₆alkyl, NR⁷R⁸, or C₁₋₄alkyl substituted with hydroxyl, halogen, or NR⁷R⁸, however, when n=0, R⁶ cannot be hydrogen;
X is C₁₋₄alkoxy or hydroxyl;
R⁷ and R⁸ are independently selected from hydrogen, C₁₋₄alkyl, or C(═O)C₁₋₆alkyl.
   Representative Examples of Preferred Novel Compounds of Formula I are:
5-(2-Aminopropyl)-8-bromo-6-methoxy-chroman-3-ol;
5-((R)-2-Aminopropyl)-8-bromo-6-methoxy-chroman-3-ol;
5-(2-Aminopropyl)-6-methoxy-8-methyl-chroman-3-ol;
5-((R)-2-Amino-1-hydroxy-propyl)-8-bromo-6-methoxy-chroman-3-ol;
Cyclopropanecarboxylic acid 5-((R)-2-aminopropyl)-8-bromo-6-methoxy-chroman-3-yl ester;
[5-(2-Aminopropyl)-6-methoxy-8-methyl-chroman-3-yl]-methanol;
5-(2-Aminopropyl)-8-iodo-chroman-3,6-diol; or
[4-(2-Aminopropyl)-5-methoxy-7-methyl-2,3-dihydro-benzofuran-2-yl]-methanol; or combinations thereof.

It is recognized that compounds of Formula I can contain one or more chiral centers. This invention contemplates all enantiomers, diastereomers and, mixtures thereof.

In the above definitions, the total number of carbon atoms in a substituent group is indicated by the Cᵢ₋ⱼ prefix where the numbers i and j define the number of carbon atoms; this definition includes straight chain, branched chain, and cyclic alkyl or (cyclic alkyl)alkyl groups. A substituent may be present either singly or multiply when incorporated into the indicated structural unit. For example, the substituent halogen, which means fluorine, chlorine, bromine, or iodine, would indicate that the unit to which it is attached may be substituted with one or more halogen atoms, which may be the same or different.

Synthesis

The compounds of Formula I can be prepared by using one of several synthetic procedures. For example, compounds of Formula I where n is 1 can be prepared from the appropriately substituted chromanols 1 as described in Scheme 1 (Chambers et al. 2001)].

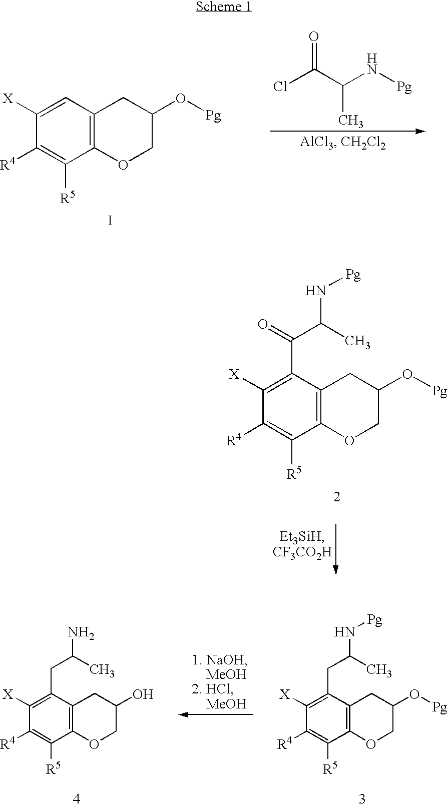

Other compounds of Formula I can be prepared from 4 through selected functional group transformations well known in the art. For example, initial protection of the primary amine group followed by activation of the hydroxyl group by formation of a sulfonate ester, e.g. methansulfonyl, and subsequent reaction with a desired nucleophile such as alkylamines, dialkylamines, aryl or alkylthiols, and the like, will provide compounds 6 of Formula I (Scheme 2). Furthermore, direct oxidation of 4 with a suitable oxidizing agent, for example, a hypervalent iodine reagent, such as o-iodoxybenzoic acid (Frigerio et al. 1995), provides the ketone 8, which can be functionalized to provide yet other compounds of Formula I, such as 9, via reductive alkylation, and 7, via Grignard addition.

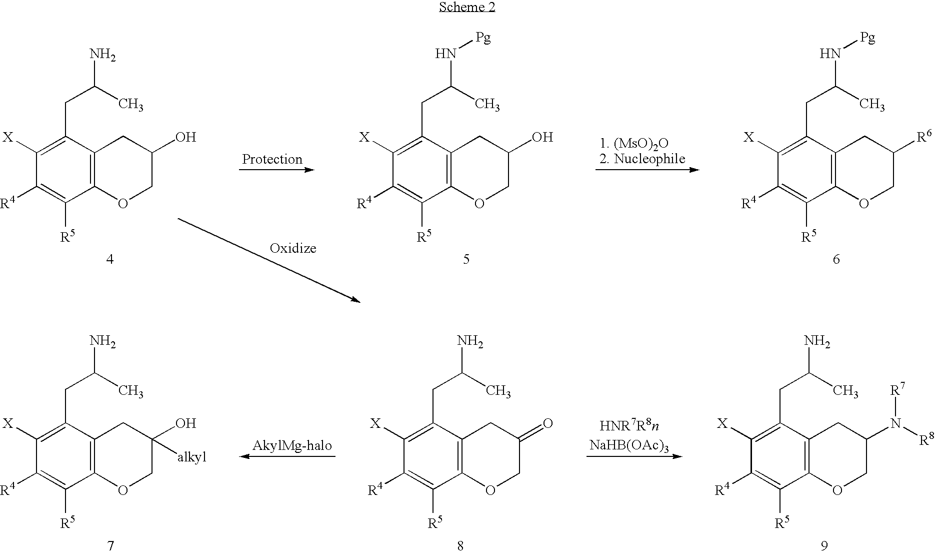

The desired chromanols 4 can be prepared from the appropriately substituted ortho-bromophenols 10, which can be purchased from commercial sources or prepared by known procedures, as described in Scheme 3. Reaction of phenols 10 with epibromohydrin using any of a variety of well known alkylation conditions, such as DMF/NaH, provides the intermediate epoxide 11. It can be advantageous to directly effect cyclization of 11 to compound 4 by treatment with a suitable base, such as n-butyllithium or under Grignard conditions. Alternately, depending on the specific substituents present, it can be more advantageous to convert 11 initially to the protected haloether 13, which will more readily undergo the cyclization reaction to provide compounds 4.

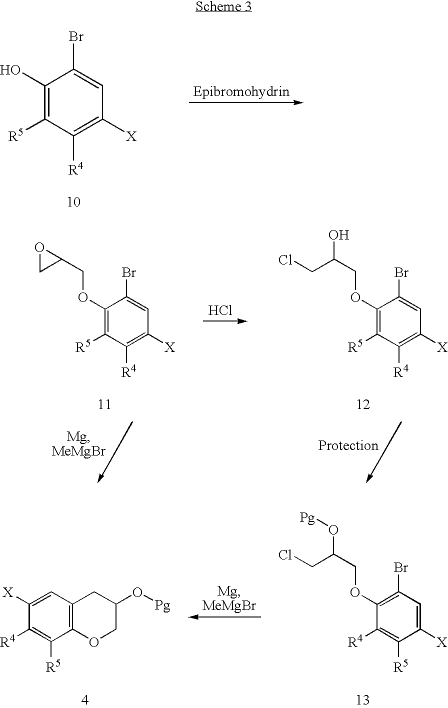

A specific example of Formula I is the preparation of the compound 21, wherein n is 1, R1, R3, and R4 are hydrogen, R2 is methyl, X is bromo, and R6 is hydroxy. This compound can be prepared from 2,6-dibromo-4-methoxyphenol (Curran et al. 1996) as described in Scheme 4.

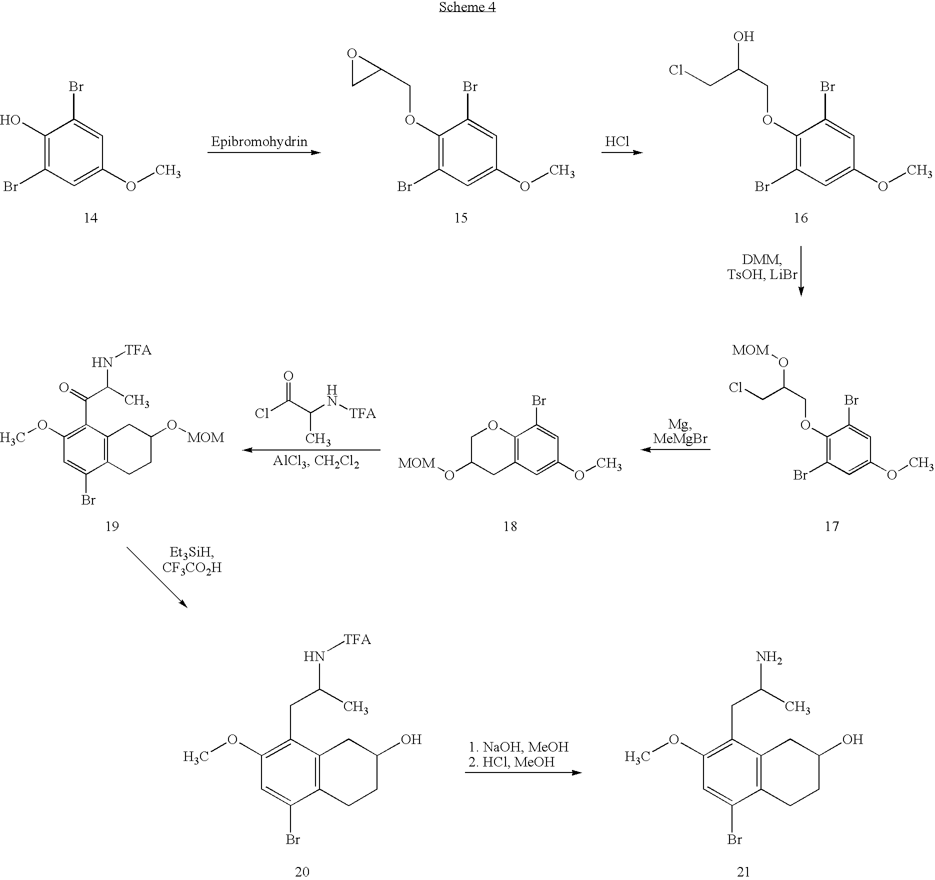

Alternately, compounds of Formula I can be prepared from propargyloxy substituted intermediates (25) via initial Claisen rearrangement reactions (Plug et al. 1992; Macor et al. 1994; Macor et al. 2000) to give the intermediate chromenes 26. Further synthetic manuplation of 26, as outlined in Scheme 5, using well-known functional group transformations provides yet other desirable compounds of Formula I.

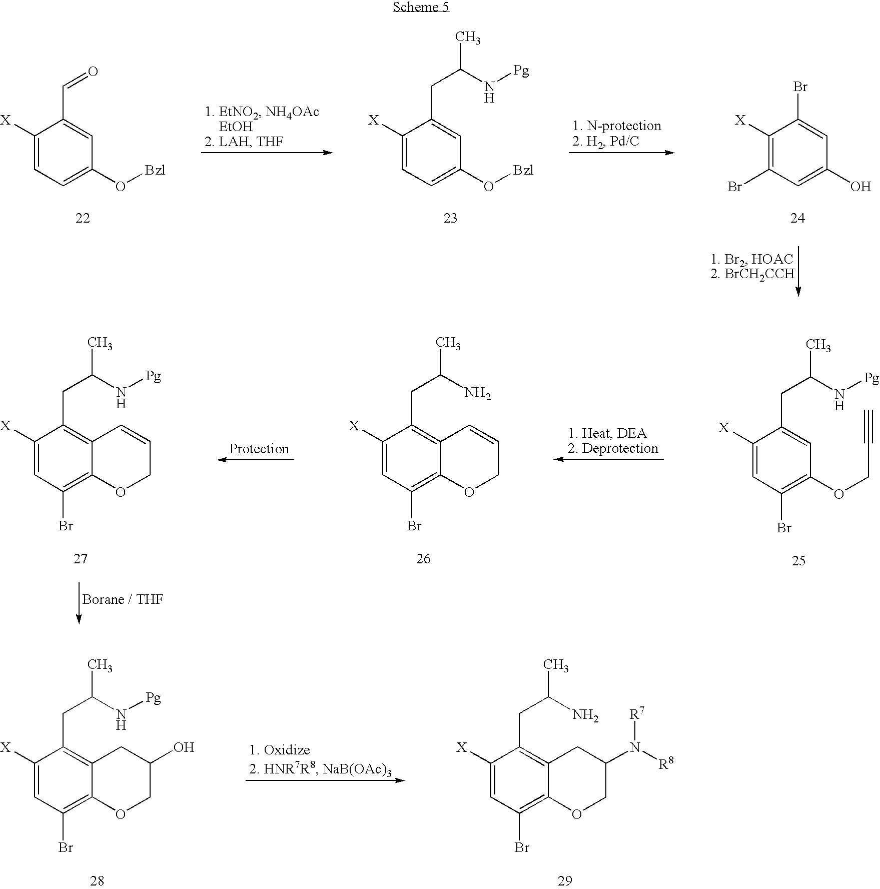

The compounds of the present invention can be used to lower and control IOP including IOP associated with normotension glaucoma, ocular hypertension, and glaucoma in warm blooded animals including humans. The compounds are preferably formulated in pharmaceutical compositions which are preferably suitable for topical delivery to the eye of the patient.

The compounds of this invention, Formula I, can be incorporated into various types of ophthalmic formulations for delivery to the eye (e.g., topically, intracamerally, or via an implant). The compounds are preferably incorporated into topical ophthalmic formulations for delivery to the eye. The compounds may be combined with ophthalmologically acceptable preservatives, viscosity enhancers, penetration enhancers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound. Furthermore, the ophthalmic solution may contain an agent to increase viscosity, such as hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-974, or the like, according to the published formulations for analogous ophthalmic preparations; preservatives and tonicity agents can be incorporated.

The compounds are preferably formulated as topical ophthalmic suspensions or solutions, with a pH of about 5 to 8. The compounds will normally be contained in these formulations in an amount 0.01% to 5% by weight, but preferably in an amount of 0.25% to 2% by weight. Thus, for topical presentation 1 to 2 drops of these formulations would be delivered to the surface of the eye 1 to 4 times per day according to the discretion of a skilled clinician.

The compounds can also be used in combination with other agents for treating glaucoma, such as, but not limited to, β-blockers (e.g., timolol, betaxolol, levobetaxolol, carteolol, levobunolol, propranolol), carbonic anhydrase inhibitors (e.g., brinzolamide and dorzolamide), α1 antagonists (e.g., nipradolol), α2 agonists (e.g. iopidine and brimonidine), miotics (e.g., pilocarpine and epinephrine), prostaglandin analogs (e.g., latanoprost, travoprost, unoprostone, and compounds set forth in U.S. Pat. Nos. 5,889,052; 5,296,504; 5,422,368; and 5,151,444, “hypotensive lipids” (e.g., bimatoprost and compounds set forth in U.S. Pat. No. 5,352,708), and neuroprotectants (e.g., compounds from U.S. Pat. No. 4,690,931, particularly eliprodil and R-eliprodil, as set forth in WO 01/85152, and appropriate compounds from WO 94/13275, including memantine.

In the formulas described above, the alkyl group can be straight-chain, branched or cyclic and the like. Halogen includes Cl, Br, F, or I. Alkoxy is understood as an alkyl group bonded through an oxygen atom.

The compounds of the present invention preferably function as 5-HT2 agonists and preferably do not enter the CNS. In more detail, the particular compounds of the present invention have incorporated into their structure a phenolic hydroxyl group which is considered comparable to that of serotonin and thus the compounds of the present invention preferably does not cross the blood-brain barrier and enter the brain. Compounds having the ability to be a 5-HT2 agonist are beneficial for controlling IOP as well as the treatment of glaucoma as shown in WO 00/16761, incorporated in its entirety by reference herein.

The compounds of the present invention preferably provide increased chemical stability and preferably achieve the desired level of therapeutic activity which includes a lowering or controlling of IOP.

The compounds of the present invention can be used in controlling or lowering IOP in warm-blooded animals including humans. Preferably, an effective amount of the compound is administered to the patient such that the IOP is controlled or lowered to acceptable levels. Furthermore, the compounds of the present invention can be used to treat glaucoma in warm-blooded animals, including humans, by administering an effective amount of the compound to a patient in need of such treatment to treat the glaucoma.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

### Method 1

### 5-HT₂ Receptor Binding Assay

To determine the affinities of serotonergic compounds at the 5-HT2 receptors, their ability to compete for the binding of the agonist radioligand [125I]DOI to brain 5-HT2 receptors is determined as described below with minor modification of the literature procedure (Johnson et al. 1987)]. Aliquots of post mortem rat or human cerebral cortex homogenates (400 μL) dispersed in 50 mM Tris-HCl buffer (pH 7.4) are incubated with [125I]DOI (80 pM final) in the absence or presence of methiothepin (10 μM final) to define total and non-specific binding, respectively, in a total volume of 0.5 mL. The assay mixture is incubated for 1 hour at 23° C. in polypropylene tubes and the assays terminated by rapid vacuum filtration over Whatman GF/B glass fiber filters previously soaked in 0.3% polyethyleneimine using ice-cold buffer. Test compounds (at different concentrations) are substituted for methiothepin. Filter-bound radioactivity is determined by scintillation spectrometry on a beta counter. The data are analyzed using a non-linear, iterative curve-fitting computer program (Bowen et al. 1995) to determine the compound affinity parameter. The concentration of the compound needed to inhibit the [125I]DOI binding by 50% of the maximum is termed the IC50 value.

### Method 2

### 5-HT₂ Functional Assay: [Ca²⁺]ᵢ Mobilization

The receptor-mediated mobilization on intracellular calcium ([Ca2+]i) was studied using the Fluorescence Imaging Plate Reader (FLIPR) instrument. Rat vascular smooth muscle cells, A7r5, were grown in a normal media of DMEM/10% FBS and 10 μg/mL gentamycin. Confluent cell monolayers were trypsinized, pelleted, and re-suspended in normal media. Cells were seeded in a 50 μL volume at a density of 20,000 cells/well in a black wall, 96-well tissue culture plate and grown for 2 days.

On the day of the experiment, one vial of FLIPR Calcium Assay Kit dye was re-suspended in 50 mL of a FLIPR buffer consisting of Hank's Balanced Salt Solution (HBSS), 20 mM HEPES, and 2.5 mM probenecid, pH 7.4. Cells were loaded with the calcium-sensitive dye by addition of an equal volume (50 μL) to each well of the 96-well plate and incubated with dye for 1 h at 23° C.

Typically, test compounds were stored at 25 μM in 50% DMSO/50% Ethanol solvent. Compounds were diluted 1:50 in 20% DMSO/20% Ethanol. For “hit” screening, compounds were further diluted 1:10 in FLIPR buffer and tested at a final concentration of 10 μM. For dose-response experiments, compounds were diluted 1:50 in FLIPR buffer and serially diluted 1:10 to give a 5- or 8-point dose-response curve.

The compound plate and cell plate were placed in the FLIPR instrument. At the beginning of an experimental run, a signal test was performed to check the basal fluorescence signal from the dye-loaded cells and the uniformity of the signal across the plate. The basal fluorescence was adjusted between 8000-12000 counts by modifying the exposure time, the camera F-stop, or the laser power. Instrument settings for a typical assay were the following: laser power 0.3-0.6 W, camera F-stop F/2, and exposure time 0.4 sec. An aliquot (25 μL) of the test compound was added to the existing 100 μL dye-loaded cells at a dispensing speed of 50 μL/sec. Fluorescence data were collected in real-time at 1.0 sec intervals for the first 60 secs and at 6.0 sec intervals for an additional 120 secs. Responses were measured as peak fluorescence intensity minus basal and where appropriate were expressed as a percentage of a maximum 5-HT-induced response. When the compounds were tested as antagonists against 10 μM 5-HT, they were incubated with the cells for 15 minutes prior to the addition of 5-HT.

The above procedures were used to generate the data shown in Table 1.

TABLE 1
5-HT_{2A} Receptor Binding and Functional Data

| | | | Efficacy |
|---|---|---|---|
| Compound | IC₅₀, nM | EC₅₀, nM | (Eₘₐₓ, %) |
| 26 | 0.31 | 108 | 23 |
| DOI | 0.33 | 30.2 | 31 |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

UNITED STATES PATENTS AND APPLICATIONS
U.S. Pat. No. 4,690,931
U.S. Pat. No. 5,151,444
U.S. Pat. No. 5,296,504
U.S. Pat. No. 5,352,708
U.S. Pat. No. 5,422,368
U.S. Pat. No. 5,494,928
U.S. Pat. No. 5,571,833
U.S. Pat. No. 5,578,612
U.S. Pat. No. 5,874,477
U.S. Pat. No. 5,889,052
U.S. Pat. No. 5,902,815
U.S. Pat. No. 5,981,572
U.S. Pat. No. 5,365,598
U.S. Pat. No. 5,380,238
U.S. Pat. No. 5,433,175

### FOREIGN PATENTS AND PUBLISHED PATENT APPLICATIONS

EP 0771563 A2
PCT/US99/19888
WO 01/85152
WO 92/0338
WO 94/13275
WO 97/35579
WO 98/18458
WO 98/31354
WO 00/16761
WO 00/35922
WO 00/44737
WO 00/44753
WO 00/77002
WO 00/77010
WO 01/12602

### OTHER PUBLICATIONS

W. P. Bowen et al., Trends Pharmacol. Sci. 16:413 (1995).
J. J. Chambers et al., J. Med. Chem. 44:1003 (2001).
D. Curran et al., J. Amer. Chem. Soc. 118:3142 (1996).
D. Fiorella et al., Psychopharmacology 121:357 (1995).
M. Frigerio et al., J. Org. Chem. 60:7272 (1995).
M. P. Johnson et al., Neuropharmacology 26:1803 (1987).
J. E. Macor et al., Tetrahedron Lett. 35:45 (1994).
J. E. Macor, Tetrahedron Lett. 41:3541 (2000).
A. P. Monte et al., J. Med. Chem. 39:2953 (1996).
A. P. Monte et al., J. Med. Chem. 40:2997 (1997).
D. E. Nichols et al., J. Med. Chem. 34:276 (1991).
M. A. Parker et al., J. Med. Chem. 41:5148 (1998).
J. P. M. Plug et al., Tetrahedron Lett. 33:2179 (1992).
Osborne et al., Ophthalmologia 210:308-314 (1996).
S. R. Waldman et al., Tetrahedron Lett. 37:7889 (1996).
Wang et al., Current Eye Research 16(8):769-775 (1997).
Wang et al., IVOS 39(4):S488 (1998).
M. S. Whiteside et al., Bioorg. Med. Chem. 10:3301 (2002).

1. A compound represented by Formula I. 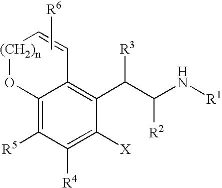
wherein R¹ is hydrogen or an alkyl group;
R² is hydrogen, an alkyl group, or R¹ and R² represent (CH₂)₂₋₄ to complete a heterocyclic ring;
R³ is hydrogen, hydroxyl, an alkoxy group, or halogen;
R⁴ and R⁵ are independently selected from hydrogen, halogen, nitrile, an alkoxy group, an alkylthiol, a substituted or unsubstituted alkyl group, or R⁴ and R⁵ represent (CH₂)ₘ to complete a cycloalkyl ring, or R⁴ and R⁵ represent or complete a phenyl or thiophene ring which is unsubstituted or substituted with halogen, an alkyl group, or an alkoxy group;
m=3-4;
n=0-2;
R⁶ is hydrogen, hydroxyl, an alkoxy group, alkoxy substituted with hydroxyl, halogen, or NR⁷R⁸, OC(═O)alkyl, ═O, NR⁷R⁸, or a substituted or unsubstituted alkyl group, wherein when n=0, R⁶ is not hydrogen;
X is an alkoxy group, hydroxyl or halogen;
R⁷ and R⁸ are independently selected from hydrogen, an alkyl group, or C(═O)alkyl;
or pharmaceutically acceptable salts or solvates thereof.

2. The compound of claim 1, wherein R¹ is hydrogen or C₁₋₄alkyl;
R² is hydrogen, C₁₋₄alkyl, or R¹ and R² represent (CH₂)₂₋₄ to complete a heterocyclic ring;
R³ is hydrogen, hydroxyl, C₁₋₄alkoxy, or halogen;
R⁴ and R⁵ are, independently selected from hydrogen, halogen, nitrile, C₁₋₄alkoxy, C₁₋₆alkylthiol, C₁₋₄alkyl, C₁₋₄alkyl substituted with halogen or C₁₋₆alkoxy, or R⁴ and R⁵ represent (CH₂)ₘ to complete a cycloalkyl ring, or R⁴ and R⁵ together complete a phenyl or thiophene ring which is unsubstituted or substituted with halogen, C₁₋₄alkyl, or C₁₋₄alkoxy;
m=3-4;
n=0-2;
R⁶ is hydrogen, hydroxyl, C₁₋₄alkoxy, C₁₋₄alkoxy substituted with hydroxyl, halogen, or NR⁷R⁸, OC(═O)C₁₋₆alkyl, ═O, NR⁷R⁸, C₁₋₄alkyl, or C₁₋₄alkyl substituted with hydroxyl, halogen, or NR⁷R⁸, wherein when n=0, R⁶ is not hydrogen;
X is C₁₋₄alkoxy or hydroxyl;
R⁷ and R⁸ are independently selected from hydrogen, C₁₋₄alkyl, or C(═O)C₁₋₆alkyl;
or pharmaceutically acceptable salts or solvates thereof.

3. The compound of claim 1, wherein said R² is hydrogen or C₁₋₄alkyl.

4. The compound of claim 1, wherein R¹ and R³ are hydrogen;
R² is C₁₋₄alkyl;
R⁴ and R⁵ are independently selected from halogen, nitrile, C₁₋₄alkoxy, C₁₋₆alkylthiol, C₁₋₄alkyl, C₁₋₄alkyl substituted with halogen, or R⁴ and R⁵ together represent (CH₂)ₘ to complete a cycloalkyl ring, or R⁴ and R⁵ together complete a phenyl or thiophene ring which is unsubstituted or substituted with halogen, C₁₋₄alkyl;
m=3-4;
n=1;
R⁶ is hydroxyl, C₁₋₄alkoxy, C₁₋₄alkoxy substituted with hydroxyl, halogen, or NR⁷R⁸, OC(═O)C₁₋₆alkyl, NR⁷R⁸, or C₁₋₄alkyl substituted with hydroxyl, halogen, or NR⁷R⁸, wherein when n=0, R⁶ is not hydrogen;
X is C₁₋₄alkoxy or hydroxyl;
R⁷ and R⁸ are independently selected from hydrogen, C₁₋₄alkyl, or C(═O)C₁₋₆alkyl.

5. The compound of claim 1, wherein said compound is:
5-(2-Aminopropyl)-8-bromo-6-methoxy-chroman-3-ol;
5-((R)-2-Aminopropyl)-8-bromo-6-methoxy-chroman-3-ol;
5-(2-Aminopropyl)-6-methoxy-8-methyl-chroman-3-ol;
5-((R)-2-Amino-1-hydroxy-propyl)-8-bromo-6-methoxy-chroman-3-ol;
Cyclopropanecarboxylic acid 5-((R)-2-aminopropyl)-8-bromo-6-methoxy-chroman-3-yl ester;
[5-(2-Aminopropyl)-6-methoxy-8-methyl-chroman-3-yl]-methanol;
5-(2-Aminopropyl)-8-iodo-chroman-3,6-diol; or
[4-(2-Aminopropyl)-5-methoxy-7-methyl-2,3-dihydro-benzofuran-2-yl]-methanol;
or combinations thereof.

6. The compound of claim 1, wherein said X is hydroxyl.

7. The compound of claim 1, wherein said X is an alkoxy group.

8. A method of controlling normal or elevated intraocular pressure comprising administering a pharmaceutically effective amount of a composition comprising at least one compound of claim 1.

9. The method of claim 8, wherein R¹ is hydrogen or C₁₋₄alkyl;
R² is hydrogen, C₁₋₄alkyl, or R¹ and R² represent (CH₂)₂₋₄ to complete a heterocyclic ring;
R³ is hydrogen, hydroxyl, C₁₋₄alkoxy, or halogen;
R⁴ and R⁵ are independently selected from hydrogen, halogen, nitrile, C₁₋₄alkoxy, C₁₋₆alkylthiol, C₁₋₄alkyl, C₁₋₄alkyl substituted with halogen or C₁₋₆alkoxy, or R⁴ and R⁵ represent (CH₂)ₘ to complete a cycloalkyl ring, or R⁴ and R⁵ together complete a phenyl or thiophene ring which is unsubstituted or substituted with halogen, C₁₋₄alkyl, or C₁₋₄alkoxy;
m=3-4;
n=0-2;
R⁶ is hydrogen, hydroxyl, C₁₋₄alkoxy, C₁₋₄alkoxy substituted with hydroxyl, halogen, or NR⁷R⁸, OC(═O)C₁₋₆alkyl, ═O, NR⁷R⁸, C₁₋₄alkyl, or C₁₋₄alkyl substituted with hydroxyl, halogen, or NR⁷R⁸, wherein when n=0, R⁶ is not hydrogen;
X is C₁₋₄alkoxy, hydroxyl or halogen;
R⁷ and R⁸ are independently selected from hydrogen, C₁₋₄alkyl, or C(═O)C₁₋₆alkyl;
or pharmaceutically acceptable salts or solvates thereof.

10. The method of claim 8, wherein R¹ and R³ are hydrogen;
R² is C₁₋₄alkyl;
R⁴ and R⁵ are independently selected from halogen, nitrile, C₁₋₄alkoxy, C₁₋₆alkylthiol, C₁₋₄alkyl, C₁₋₄alkyl substituted with halogen, or R⁴ and R⁵ together represent (CH₂)ₘ to complete a cycloalkyl ring, or R⁴ and R⁵ together complete a phenyl or thiophene ring which is unsubstituted or substituted with halogen, C₁₋₄alkyl;
m=3-4;
n=1;
R⁶ is hydroxyl, C₁₋₄alkoxy, C₁₋₄alkoxy substituted with hydroxyl, halogen, or NR⁷R⁸, OC(═O)C₁₋₆alkyl, NR⁷R⁸, or C₁₋₄alkyl substituted with hydroxyl, halogen, or NR⁷R⁸, wherein when n=0, R⁶ is not hydrogen;
X is C₁₋₄alkoxy or hydroxyl;
R⁷ and R⁸ are independently selected from hydrogen, C₁₋₄alkyl, or C(═O)C₁₋₆alkyl.

11. The method of claim 8, wherein said compound is:
5-(2-Aminopropyl)-8-bromo-6-methoxy-chroman-3-ol;
5-((R)-2-Aminopropyl)-8-bromo-6-methoxy-chroman-3-ol;
5-(2-Aminopropyl)-6-methoxy-8-methyl-chroman-3-ol;
5-((R)-2-Amino-1-hydroxy-propyl)-8-bromo-6-methoxy-chroman-3-ol;
Cyclopropanecarboxylic acid 5-((R)-2-aminopropyl)-8-bromo-6-methoxy-chroman-3-yl ester;
[5-(2-Aminopropyl)-6-methoxy-8-methyl-chroman-3-yl]-methanol;
5-(2-Aminopropyl)-8-iodo-chroman-3,6-diol; or
[4-(2-Aminopropyl)-5-methoxy-7-methyl-2,3-dihydro-benzofuran-2-yl]-methanol;
or combinations thereof.

12. A method for the treatment of glaucoma comprising administering a pharmaceutically effective amount of a composition comprising at least one compound of claim 1.

13. The method of claim 12, wherein R¹ is hydrogen or C₁₋₄alkyl;
R² is hydrogen, C₁₋₄alkyl, or R¹ and R² represent (CH₂)₂₋₄ to complete a heterocyclic ring;
R³ is hydrogen, hydroxyl, C₁₋₄alkoxy, or halogen;
R⁴ and R⁵ are independently selected from hydrogen, halogen, nitrile, C₁₋₄alkoxy, C₁₋₆alkylthiol, C₁₋₄alkyl, C₁₋₄alkyl substituted with halogen or C₁₋₆alkoxy, or R⁴ and R⁵ represent (CH₂)ₘ to complete a cycloalkyl ring, or R⁴ and R⁵ together complete a phenyl or thiophene ring which is unsubstituted or substituted with halogen, C₁₋₄alkyl, or C₁₋₄alkoxy;
m=3-4;
n=0-2;
R⁶ is hydrogen, hydroxyl, C₁₋₄alkoxy, C₁₋₄alkoxy substituted with hydroxyl, halogen, or NR⁷R⁸, OC(═O)C₁₋₆alkyl, ═O, NR⁷R⁸, C₁₋₄alkyl, or C₁₋₄alkyl substituted with hydroxyl, halogen, or NR⁷R⁸, wherein when n=0, R⁶ is not hydrogen;
X is C₁₋₄alkoxy, hydroxyl or halogen;
R⁷ and R⁸ are independently selected from hydrogen, C₁₋₄alkyl, or C(═O)C₁₋₆alkyl;
or pharmaceutically acceptable salts or solvates thereof.

14. The method of claim 12, wherein R¹ and R³ are hydrogen;
R² is C₁₋₄alkyl;
R⁴ and R⁵ are independently selected from halogen, nitrile, C₁₋₄alkoxy, C₁₋₆alkylthiol, C₁₋₄alkyl, C₁₋₄alkyl substituted with halogen, or R⁴ and R⁵ together represent (CH₂)ₘ to complete a cycloalkyl ring, or R⁴ and R⁵ together complete a phenyl or thiophene ring which is unsubstituted or substituted with halogen, C₁₋₄alkyl;
m=3-4;
n=1;
R⁶ is hydroxyl, C₁₋₄alkoxy, C₁₋₄alkoxy substituted with hydroxyl, halogen, or NR⁷R⁸, OC(═O)C₁₋₆alkyl, NR⁷R⁸, or C₁₋₄alkyl substituted with hydroxyl, halogen, or NR⁷R⁸, wherein when n=0, R⁶ is not hydrogen;
X is C₁₋₄alkoxy or hydroxyl;
R⁷ and R⁸ are independently selected from hydrogen, C₁₋₄alkyl, or C(═O)C₁₋₆alkyl.

15. The method of claim 12, wherein said compound is:
5-(2-Aminopropyl)-8-bromo-6-methoxy-chroman-3-ol;
5-((R)-2-Aminopropyl)-8-bromo-6-methoxy-chroman-3-ol;
5-(2-Aminopropyl)-6-methoxy-8-methyl-chroman-3-ol;
5-((R)-2-Amino-1-hydroxy-propyl)-8-bromo-6-methoxy-chroman-3-ol;
Cyclopropanecarboxylic acid 5-((R)-2-aminopropyl)-8-bromo-6-methoxy-chroman-3-yl ester;
[5-(2-Aminopropyl)-6-methoxy-8-methyl-chroman-3-yl]-methanol;
5-(2-Aminopropyl)-8-iodo-chroman-3,6-diol; or
[4-(2-Aminopropyl)-5-methoxy-7-methyl-2,3-dihydro-benzofuran-2-yl]-methanol;
or combinations thereof.

16. A pharmaceutical composition comprising the compound of claim 1 and at least one carrier.

17. A method to block or bind to serotonin receptors comprising administering an effective amount of at least one compound of claim 1 to a patient.
